(19) United States
(12) Patent Application Publication
(10) Pub. No.: US 2023/0233745 A1
(43) Pub. Date: Jul. 27, 2023

(54) PUMP HAVING ELECTROACTIVE POLYMERS AND A RETURN ELEMENT

(71) Applicant: FRESENIUS MEDICAL CARE DEUTSCHLAND GMBH, Bad Homburg (DE)

(72) Inventors: Gerome NEWPORT FISCHER, Weberstedt (DE); Tobias STÖCKERL, Tiefenbach (DE); Jonas HELLHUND, Frankfurt a. Main (DE)

(73) Assignees: FRESENIUS MEDICAL CARE DEUTSCHLAND GMBH, Bad Homburg (DE); FRESENIUS MEDICAL CARE AG & Co. KGaA, Bad Homburg (DE)

(21) Appl. No.: 18/003,556

(22) PCT Filed: Jun. 30, 2021
(86) PCT No.: PCT/EP2021/068056

§ 371 Date: Dec. 28, 2022

(30) Foreign Application Priority Data
Jun. 30, 2020 (DE) 10 2020 117 221.4

(51) Int. Cl. A61M 1/16; F04B 43/04

(52) U.S. Cl. CPC A61M 1/16; F04B 43/04

(57) ABSTRACT

The present invention relates to a pump, comprising a pump actuator (2) having at least one electroactive polymer, as well as at least one return element (8, 9) which returns a displacement element (5) of the pump after a pump stroke to a defined position. The invention further relates to a metering unit and to a medical apparatus comprising such a pump.

The present invention relates to a pump having a pump actuator having at least one electroactive polymer, wherein the at least one electroactive polymer is arranged such that a deformation of the at least one electroactive polymer takes place along the direction of the pump stroke of the pump.

The present invention further relates to a blood treatment machine having such a pump. In addition a pump in accordance with the invention can inter alia be used as part of a pressure retention test in the context of a blood treatment machine.

The required solutions are in particular prepared from concentrates, that are diluted as required, during an ongoing treatment as part of modern extracorporeal blood treatments. Pumps are used in this respect that meter the required concentrate amounts to provided dialysis water.

Particularly accurate, finely controllable pumps are required so that the solutions produced correspond to the objectives as accurately as possible. It is additionally necessary to be able to reproduce and optionally to document the pump strokes carried out by a pump that is used in order thus to be able to detect the amounts and volumes conveyed by the pump as accurately as possible.

Pumps having electroactive polymers can be used, for example. However, so-called creeping or hysteresis occurs on the use of such polymers. This is in particular the case with electroactive polymers since a main component thereof is a layer of silicone or of a similar material.

On an operation of a pump having an electroactive polymer at a regular and typically very high frequency, this hysteresis has the effect that no complete return of the polymer and thus of the membrane or of the piston of the pump takes place. This has consequences, on the one hand, for the conveyed volume, it becomes smaller; it also has consequences for the maximum possible pressure toward the end of the pump stroke, it also becomes smaller.

It is the underlying object of the present invention to alleviate or even fully eliminate the problems known from the prior art. It is in particular the object of the invention to provide a particularly accurate pump.

This object is achieved by a pump in accordance with claim 1. A further aspect of the invention relates to a metering unit and to a blood treatment machine having a pump in accordance with the invention.

A pump in accordance with the invention for a medical device, in particular a blood treatment device, preferably a dialysis machine, has the following components: a pump actuator having at least one electroactive polymer, wherein the pump actuator has a trigger element, a displacement element, and a return element, with the return element being configured such that the displacement element can be returned to a defined position after an actuation of the trigger element. The above-described pump can here also be used as a valve or can be configured as a valve.

A valve in accordance with the invention for a medical device, in particular a blood treatment device, preferably a dialysis machine, can thus have the following components: a pump actuator having at least one electroactive polymer, wherein the pump actuator has a trigger element, a displacement element, and a return element, with the return element being configured such that the displacement element can be returned or brought to a defined position after an actuation of the trigger element.

The defined position here can be a third position that corresponds to the first position, the starting position. The second position that is reached after the first position and before the third position can thus be a position that differs from the pump position. The defined position is, for example on the use of EAP, more accurately on the use of a plurality of layers, preferably a stack, of electroactive polymers, in particular dielectrically electroactive polymers, as the displacement element, is the first position, the pump position. It can additionally be achieved that a provided deflection in the pump direction or the displacement direction is always reached by the return element. In this case, the displacement element and the trigger element can be the EAP, that is the electroactive polymer. It is thus possible to counteract hysteresis by the return element. In other words, it can be ensured that the pump position is reached in every pump process. The pump position here is the starting position in which the pump actuator is returned by the return element. The pump actuator reaches the “volume position” differing from the pump position after the triggering of the trigger element. In other words, the second position (volume position) can be a position that is furthest away from the pump position and thus, for example, enables a topping up of fluids. If the first or third position, that is the pump position, is subsequently reached again, the fluid can thus be displaced. In other words, the defined position can be a pump position, the displacement element and the trigger element can be configured as electroactive polymers, and the return element can be moved fully into the defined position or defined pump position. The return element can here act on the displacement element in the direction of the pump position.

The defined position can equally be a third position that corresponds to the first position, the starting position. This third position is reliably reached by means of the return element, for example, on the use of electrodes arranged in ring form, that is on the use of COP-DEA (circular out-of-plane dielectric elastomer actuator). The starting position, that is the position before the triggering of the trigger element, is thus reached by the return element after the triggering and after the pump procedure.

The displacement element can in this case be a spring, a magnet, or a capacitor. The trigger element can in this base be the COP-DEA, that is the electrode arranged in ring form, preferably having a respective silicone layer disposed between the electrodes. The return element can in this case be a spring, a magnet, or a capacitor. In other words, the defined position can be a starting position that differs from the pump position, the displacement element can differ from the trigger element, with the trigger element being configured as a COP-DEA, and the return element can fully move the displacement element into the defined position or defined starting position. The return element here can move the displacement element fully into the defined position by means of the trigger element.

The pump actuator can thus also be understood as or called a valve actuator.

The return element preferably has at least one spring, magnet or capacitor. The return element can preferably move the trigger element into a pump position that corresponds to the starting position. In this respect, the return element acts against the deflection direction that is applied by the trigger element. In other words, the pump actuator is moved out of the pump position by the trigger element and is subsequently moved back fully into the pump position by the return element. The return element can alternatively move the trigger element into a third position that corresponds to the starting position. In this case, the pump position can be adopted after the triggering of the trigger element. It is ensured by the return element that the pump actuator reaches the starting position that differs from the pump position in this case.

It has furthermore proved advantageous for the pump actuator or valve actuator, preferably the displacement element and/or the trigger element, to have a plurality of layers, preferably a stack, of electroactive polymers, in particular dielectric electroactive polymers.

The pump actuator or valve actuator preferably has electrodes arranged in ring form, respectively having a silicone layer disposed between the electrodes. These electrodes arranged in ring form are preferably configured as the trigger element here.

Provision can furthermore be made that the pump actuator has a membrane and the displacement element has a plurality of layers, preferably a stack, of electroactive polymers, and the membrane can be moved from a starting position into a pump position by means of the electroactive polymers.

It has furthermore proved advantageous for the trigger element to have a plurality of layers, preferably a stack, of electroactive polymers or electrodes arranged in ring form, respectively having a silicone layer disposed between the electrodes.

A further embodiment of the invention relates to a pump or valve, with the displacement element and/or the return element and/or the trigger element being formed as one element in part.

The return element preferably has one or more magnets having at least 8 individual poles, preferably more than 10 individual poles, more preferably more than 14 individual poles.

It has furthermore proved advantageous in practice for the return element to comprise at least one capacitor, with the capacitor being charged by the released energy on a dilatation of the electroactive polymer and with the released energy being able to be used for a contraction of the electroactive polymer on a discharge of the capacitor. The dilatation of the electroactive polymer occurs when the electroactive polymer—itself a kind of capacitor—is discharged. The electrical energy from the electroactive polymer is therefore wholly or partially displaced into the capacitor included in the return element. The electroactive polymer, that can be considered a capacitor, is discharged here and the capacitor of the return element is charged—or vice versa.

A further aspect of the invention relates to a pump in accordance with the invention or a valve, with the return element being a capacitor, the trigger element being configured as an electroactive polymer in the form of a dielectric elastomer, in particular a stack of electroactive polymers, and the displacement element being at least one spring, at least one magnet, or at least one capacitor, each being connected to a movable membrane, preferably further comprising an electric energy storage device that can store the energy of the capacitor and/or of the dielectric elastomer. In a variant, one or more layers of the stack of the electroactive polymer form a part of a capacitor of a return element. This layer/these layers is/are preferably arranged at the end of the stack that is located closest to the top of the pump space or valve space.

The following special advantages result for variants of the invention in which the return element comprises at least one capacitor. The return force can be particularly advantageously finely controlled and even cancelled in comparison with return element having magnets or springs. The return force can be controlled by a control of the voltage applied to the capacitor. It particularly advantageously results therefrom that the return force does not have to be overcome, for example when the membrane moves away from the upper end abutment. Further advantages thereby result such as smaller mechanical demands on the pump actuator and the pump overall. Energy is saved when work does not have to be done against the return element. A required return can be detected by the measurement of the electrical properties of the capacitor. This variant is accordingly capable of a further expanded self sensing, namely the reaching of the upper end abutment of the pump. The advantage further results that the variant can pump faster since the resistance of a return element in the opening cycle does not have to be overcome. The charge in one half of the capacitor can furthermore be particularly advantageously exchanged by an uneven charge so that the capacitor plates repel one another and the return element does not return in this situation, but rather assists the removal of the membrane from the upper end abutment. A variable return element is thus provided that can support a “closing process” or an “opening process” by an electric motor in an attractive and repulsive state. This assistance by an electric motor can be particularly advantageously accurately controlled by the voltage dependence.

Another aspect of the invention relates to a metering unit, in particular a metering unit for a blood treatment device, having a pump in accordance with the invention or a valve.

The invention moreover relates to a medical device, in particular to a blood treatment machine, having a pump and/or a metering unit in accordance with the present invention.

The invention further relates to a use of a pump in accordance with the invention on/in a medical device, in particular a blood treatment device, preferably a dialysis machine.

The pump can here also be used or configured as a valve.

Yet another aspect of the invention relates to a method of pumping fluid for a medical device, in particular a blood treatment device, in particular a dialysis machine, having a pump in accordance with the invention, with the pump having a pump space for conveying fluid, said method comprising the steps: triggering the trigger element by varying an electric voltage; moving the displacement element from a starting position into a pump position so that fluid is displaced from a pump space; returning the displacement element from the pump position into the starting position; or returning the displacement element to the pump position after leaving the starting position.

The return element here preferably has a capacitor; the triggering of the trigger element takes place by a discharge of the capacitor and a return of the displacement element takes place by a charging of the capacitor; and/or at least a portion of the electrical charge or energy of the capacitor flows between the return element and the displacement element.

A pump in accordance with the invention has a pump actuator having at least one electroactive polymer and has at least one return element that guides or pulls a displacement element back into a defined position after a pump stroke. In other words, the return element ensures the reaching of a defined position. This defined position can here correspond to the starting position or the pump position. A membrane is preferably attached to the displacement element.

The membrane can here be a separating layer that bounds a pump space here. The membrane can thus also be at least partially formed by the uppermost layer of the electroactive polymer. The membrane can equally be a film, in particular a silicone film, that is moved by the electroactive polymer.

The return element thus compensates the hysteresis in that it ensures that the displacement element of the pump is moved on every pump stroke up to a certain desired abutment or up to a desired position.

The return element thus prevents the conveyed volume from becoming smaller over time due to the hysteresis and moreover prevents a reduction of the pump pressure toward the end of each pump stroke.

The return element is preferably a spring, a magnet, or a capacitor, or also a combination of these components.

It has proved advantageous in practice for the electroactive polymer to be designed as a stack of a plurality of layers of electroactive polymers or as a circular out-of-plane polymer.

A plurality of stacks of a plurality of layers of electroactive polymers can also be provided that are arranged next to or above one another or act in series or in parallel.

Circular out-of-plane electroactive polymers (COP-EAPs or COP-DEAs) have a membrane-like appearance, are typically arranged in a rigid frame, and have a rigid region at the center.

If a voltage is applied, the stiffness of the COP changes. The COP is here formed about a first element and arranged within a second element. On a change of the stiffness of the COP, the first and second elements can thus be displaced relative to one another. This displacement is achieved in that a preload is set between the first and second elements. A spring, a magnet, or a capacitor can be used as the return element for this purpose. A spring, a magnet, or a capacitor can equally be used as the displacement element in the embodiment with a COP.

If a spring is added to this arrangement, for example, the deformation can be controlled in a specific direction and the COP polymer membrane can be used in the framework of a pump. The pump force here does not result from the EAP, but rather from the spring. The electroactive polymer in this embodiment mainly serves the controlled triggering and tensioning of the spring.

This mechanism can furthermore be combined with a bistable spring, for example having a negative gradient, to achieve a greater stroke. This bistable spring then jumps about between two stable points. A special desired characteristic thus results from the combination of the characteristics of both springs (the bistable spring and another conventional spring). In the embodiment with a COP, the return element can, as described above, be configured as a spring, a magnet, or a capacitor. The return element here acts in a direction that acts against the displacement element. In other words, the return element has the effect that the COP is again moved back to its original starting position (prior to the triggering) after the triggering, that is caused by the application of a voltage, and after the action of the displacement element. In other words, it is ensured by the return element that the COP adopts the same position that was present prior to the triggering.

In accordance with an embodiment of the invention in which the deformation of the EAP is used after the application of a voltage, the electroactive polymer (EAP) forms the displacement element. In other words, the deformation (e.g. contraction and dilatation) of e.g. a stack of electroactive polymers can be used to move a medium to be conveyed.

In this respect, the displacement element can comprise of consist of an electroactive polymer.

In accordance with another aspect of the invention, a plurality of return elements can be provided, in particular a plurality of magnets or a combination of at least one magnet and at least one spring.

The use of magnets has the advantage that the force between two magnets is the greatest when they are close to one another. If one magnet is placed, for example, directly at the displacement element or at the membrane connected to the displacement element (thereunder) and one in the top of the pump chamber, the maximum fore acts at the top dead center of the stroke movement when the membrane has approached the top of the pump chamber by a maximum. It is thus ensured that the displacement element, the EAP, reaches the second position, that is the pump position or displacement position, by means of the return element.

If the membrane should be pulled down again away from the top of the pump chamber, the force of the magnets only has to be additionally overcome at the start of the movement. The force exerted by the magnets will, however, become smaller and smaller in the following; in the best case such that the force only has to be taken into account right at the start of the stroke. It is ensured by this additional force that the membrane always contacts the top of the pump chamber and there is thus a defined abutment.

The arrangement of the magnets in a pump in accordance with the invention is arbitrary and can be adapted to the respective demands. A magnet can, for example, be alternatively or additionally provided at the base of the pump chamber so that it is ensured on every stroke that the membrane reaches an upper and a lower defined position or an abutment. It is equally conceivable to arrange magnets at one or both sides of the walls of the pump chamber.

The magnets used can satisfy different demands or be present in different designs. In the simplest case, permanent magnets such as neodymium magnets are provided. Electromagnets would also be conceivable.

It has furthermore found to be advantageous in practice for the return element to comprise at least one polymagnet (also known as multimagnets or programmable magnets).

Polymagnets have different regions of different magnetic materials and can have properties especially adapted to the respective case of use such as a desired force curve due to their magnet patterns. They can be designed, for example, such that the force is very high in direct proximity of two polymagnets to one another (for example 0.3 mm), but drops very steeply thereafter.

A polymagnet can, for example, be configured such that the field line density has reduced by 50% after 20%, preferably after 10%, of the maximum stroke of the displacement element.

A polymagnet in the sense of the invention in particular has field lines that are formed perpendicular to the disk surface. A polymagnet in particular does not have any field lines that extend radially outwardly of the magnetic disk. The polymagnet can here be formed as a circular magnetic disk.

Polymagnets can here be individual magnets that are formed in or on a substrate or are pressed into the magnetizable substrate. In this respect, the polarity and the arrangement of the magnets can be selected such that desired shear and torque characteristics are achieved. In other words, a desired polarity pattern can be achieved by the manufacturing process.

It would, for example, be conceivable that instead of a spring polymagnets are used that are provided with such a magnetic pattern that they repel at near and attract at far. A pump in accordance with the invention can be designed with this force such that the magnets attract for so long until they are in direct proximity and then repel one another again, whereby the displacement element is guided both by a respective complete pump stroke and is then again moved in the opposite direction. Such a mechanism has the advantage that it works completely without friction and wear phenomena.

In accordance with another aspect of the invention, the return element comprises at least one capacitor, with the capacitor being charged by the released energy on a dilatation of the electroactive polymer and with the released energy being able to be used for a contraction of the electroactive polymer on a discharge of the capacitor.

This procedure is also called energy harvesting and allows a particularly high energy efficiency of a pump in accordance with the invention.

Instead of magnets, capacitors can generally also be used as the return element or to generate the required attraction force to move the displacement element in a complete defined ump stroke.

The capacitors provide the possibility of energy harvesting. The contraction movements of electroactive polymers corresponds to the charging of a capacitor. The charge is again conducted from the electroactive polymer or capacitor to enable the return or dilatation of the electroactive polymer. This charge can be used to charge the capacitor used as the return element or for the attraction. If it is again discharged, the charge can again be used for a targeted contraction of the electroactive polymer.

A pump in accordance with the invention can in particular be used within the framework of an extracorporeal blood treatment. A further aspect of the invention relates, for example, to a metering unit, in particular a metering unit for a blood treatment device, having a pump in accordance with the invention.

Another aspect of the invention relates to a medical device , in particular a blood treatment machine, in particular a dialysis machine, having a pump in accordance with the invention and/or a metering unit.

The invention equally comprises the use of a pump in accordance with the invention in a medical device, in particular a blood treatment machine.

A pump in accordance with the invention preferably has a pump actuator having at least one electroactive polymer, wherein the at least one electroactive polymer is preferably arranged such that a deformation of the at least one electroactive polymer takes place along the direction of the pump stroke of the pump.

In other words, the deformation/length extension of the electroactive polymer preferably takes place along the direction of the pump movement/pump stroke of the pump. This provides the advantage that the amount of the pump stroke is directly proportional to or correlates with the amount of the deformation longitudinal extension of the electroactive polymer.

The amount of an executed pump stroke can thus be derived particularly clearly and directly from the corresponding deformation of the electroactive polymer due to the arrangement of the electroactive polymer.

Electroactive polymers deform in response to a potential that is applied to the electroactive polymer and can serve by this deformation, for example, as a pump actuator since a medium to be conveyed is pressed out of the pump by the deformation.

The impedance or another measurable property of the electroactive polymers changes here, for example, on a mechanical force application. This property of electroactive polymers, that the measurable properties vary in dependence on a mechanical force application, is also called “self sensing” and makes it possible to use electroactive polymers as sensors to determine the forces that act and thus, for example, the height of the pump strokes using e.g. the measured impedance or another measurable property. The at least one electroactive polymer of a pump in accordance with the invention preferably has self sensing properties. How large every executed pump stroke was can be determined directly with a pump in accordance with the invention by means of the self sensing properties.

Such an accurate monitoring capability of the conveyed volumes is in particular essential with metering pumps that are used within the framework of a blood treatment.

It has proven advantageous in practice for the at least one electroactive polymer to form a pump actuator of the pump. The electroactive polymer can be deformed by the application of a potential or of a voltage to it, whereby medium to be conveyed is moved into the pump since the deformation/longitudinal extension of the electroactive polymer takes place along the direction of the pump movement/pump stroke of the pump. Medium to be conveyed can thereupon be pressed out of the pump due to a relaxation of the electroactive polymer.

The electroactive polymer can preferably drive a piston or a pump membrane or can also be in direct contact with the medium to be conveyed.

With a pump in accordance with the invention, a plurality of layers of electroactive polymers are preferably provided that are preferably arranged as a stack. Alternatively or additionally, a plurality of such stacks of a plurality of layers of electroactive polymers can also be arranged next to and/or above one another.

A pump in accordance with the invention preferably further comprises a control unit that controls the at least one electroactive polymer for a defined deformation in order to thus actuate the pump. If a plurality of stacks are provided, every stack can be equipped with its own control unit, but only one common control unit can also be provided.

A pump in accordance with the invention preferably furthermore comprises a measuring unit that measures the deformation of the at least one electroactive polymer and/or the distance of at least two layers of a stack of electroactive polymers to thus determine the degree of deformation or the amplitude of a pump stroke of the pump.

The measurement unit can be part of the control unit and/or can exchange data therewith. Alternatively or additionally, the measuring device can be adapted to transmit the measured data to an external receiver so that the function of the ump can be monitored and/or documented remotely.

In addition, the pump can be equipped with a force bundling structure that is adapted to bundle the forces arising from the deformation of the at least one electroactive polymer and transmit them directly to another structure, with the area of the force bundling structure preferably being smaller than the area of the at least one electroactive polymer.

The forces/pressure of the electroactive polymer or the stack of electroactive polymers is/are focused by the reduction of the area so that a higher pressure can be reached. If the force bundling structure presses onto a valve seat, for example, a particularly safe closing of the pump can be hereby achieved, for example.

For example, two stacks of electroactive polymers arranged next to or above one another can act on a common force bundling structure.

The at least one electroactive polymer can furthermore be connected to a pressure transducer with a pump in accordance with the invention. The pressure transducer can here likewise be a stack of electroactive polymers, whereby the self sensing properties can be amplified and the actuation of the pump (pump stroke, frequency, etc.) can be monitored particularly precisely.

The at least one electroactive polymer can be connected to the force bundling structure by the pressure transducer and/or the pressure transducer can act as the force bundling structure.

A further aspect of the invention relates to a metering unit, preferably to metering unit of a blood treatment machine, having at least one pump in accordance with the invention. The metering unit furthermore has valves to control the flow of fluids such as concentrates or dialysis water. The metering unit additionally comprises a control unit by means of which the valves can be controlled.

A further aspect of the invention relates to a blood treatment machine, in particular a dialysis machine, having at least one pump in accordance with the invention and/or a metering unit in accordance with the invention.

In accordance with an embodiment of the invention, a blood treatment machine has at least two pumps in accordance with the invention, wherein a first pump meters or feeds fluid, preferably concentrates, downstream of a balancing device and a second pump removes fluid, preferably liquid, upstream of the balancing device so that the fluid balance determined in the balancing device remains unchanged.

Alternatively or additionally, the two pumps in accordance with the invention can be arranged upstream and/or downstream of a dialyzer and/or of a dialysis liquid filter.

To balance differences of the pumps used caused by the production, provision can advantageously be made that the first and second pumps swap their functions at predetermined points in time/intervals. The pumps and/or the control unit is/are thus preferably adapted to swap the function of the pumps.

In accordance with an embodiment of the invention, the at least two pumps are furthermore adapted to additionally carry out an ultrafiltration. In this embodiment, additional valves have to be provided that enable the pumps to carry out an ultrafiltration. An ultrafiltration pump can, however, be omitted in this embodiment.

A further aspect of the invention relates to a use, for example an installation, of a pump in accordance with the invention in a blood treatment device, for example a dialysis machine.

A (single) pump in accordance with the invention can, for example, preferably be used as part of a metering of concentrates by a blood treatment device to meter a plurality of different concentrates or solutions such as acid concentrate and sodium bicarbonate. This is possible due to the achievable high pump frequency of a pump in accordance with the invention. A single pump in accordance with the invention could thus replace two conventional membrane pumps.

In this application, the pump in accordance with the invention is preferably equipped with two lines (first line, e.g. acid concentrate, and second line, sodium bicarbonate) or with three lines (first line, e.g. acid concentrate, second line, sodium bicarbonate, third line, flushing solution). The first solution and the second solution are preferably pumped alternately, flushing can take place therebetween.

The operation of a pump in accordance with the invention for the alternating conveying of different solutions is a further aspect of the invention. Such an operating procedure can be stored in a corresponding control unit, for example. In accordance with an advantageous embodiment, a blood treatment machine in accordance with the invention has a first pump in accordance with the invention that meters in at least two different solutions and that is preferably arranged downstream of a balancing device and has a first pump in accordance with the invention that removes liquid to the same degree and is preferably arranged upstream of the balancing device.

Another aspect of the invention relates to a method of measuring a pressure maintenance in a filter, in particular in a filter for filtering dialysis liquid or in a dialyzer, with a pump in accordance with the invention being used as part of the method.

In the method in accordance with the invention for measuring a pressure maintenance in a filter, the self sensing property of the electroactive polymer is used.

A defined fluid volume is, for example, introduced into the filter to be tested. The defined fluid volume in the filter generates a specific internal pressure in the filter that acts on the electroactive polymer in the pump.

In response to the pressure, the electroactive polymer generates a corresponding electric potential that can be detected by means of a measurement unit. Any pressure loss in the filter, due to a leak, for example, is thus mirrored in the measured potential profile of the electroactive polymer and can thus be detected. If the potential and thus the pressure in the filter remains constant, the intactness of the filter can be concluded.

As previously described, the pump can be configured as a valve in the previously described aspects.

Further features, advantages, and effects of the present invention result from the following description of preferred embodiments of the invention with reference to the associated Figures in which similar or the same components are marked by the same reference numerals.

There are shown:

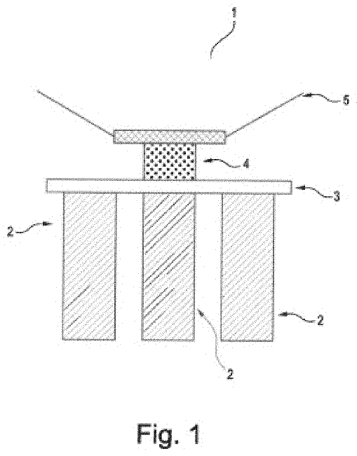

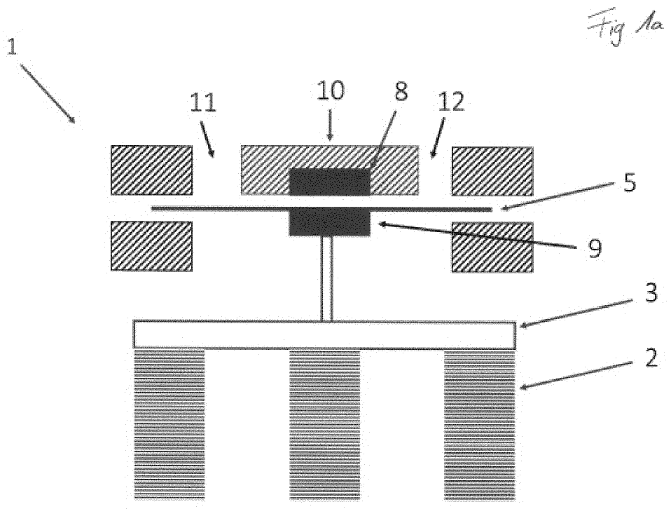

FIG. 1 a schematic drawing of an embodiment of a pump in accordance with the invention;

FIG. 1a a schematic drawing of a different embodiment of a pump in accordance with the invention;

FIG. 1b a schematic drawing of a further embodiment of a pump in accordance with the invention;

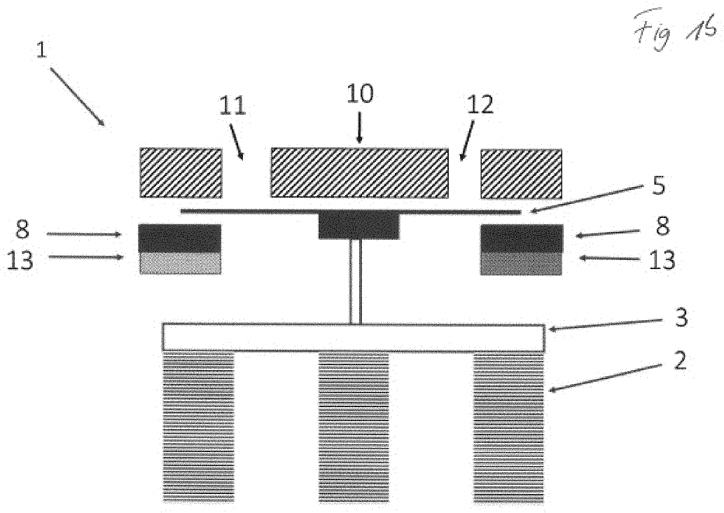

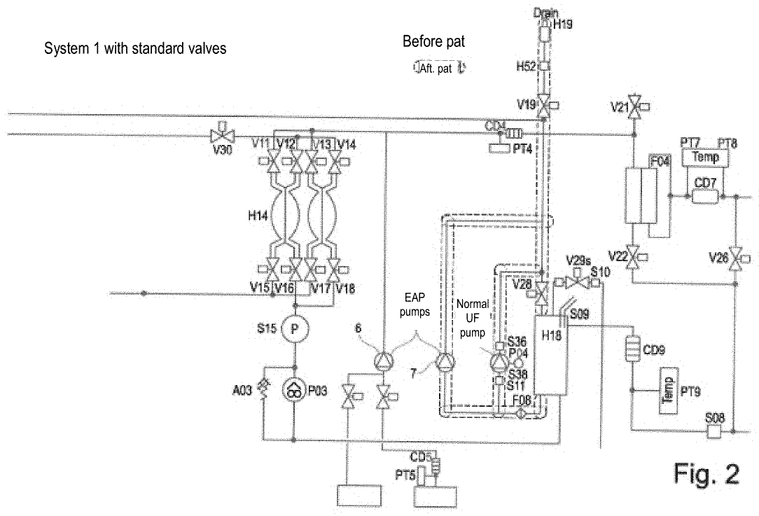

FIG. 2a flowchart that illustrates the use of pumps in accordance with the invention as part of an embodiment of a blood treatment machine;

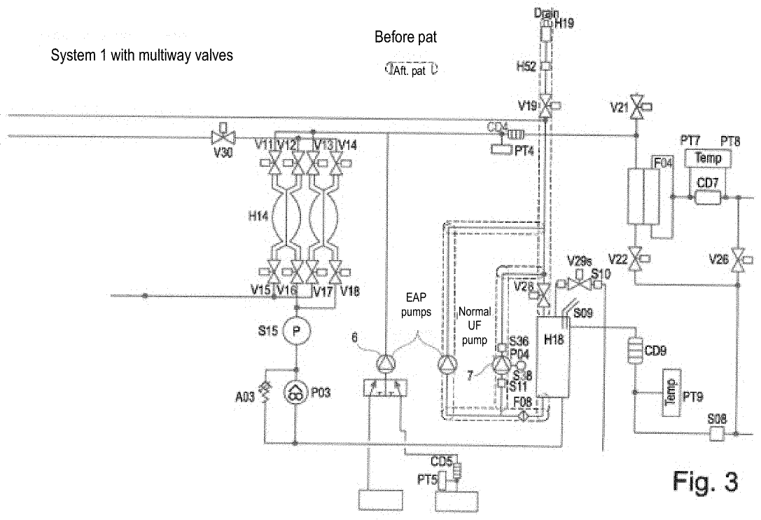

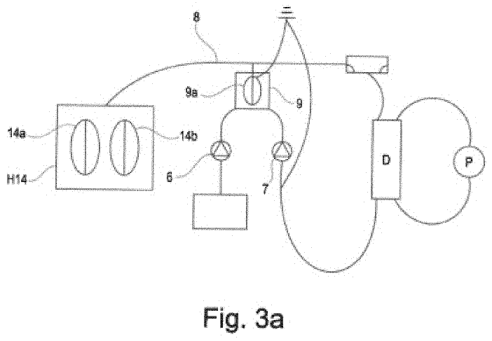

FIG. 3 the embodiment of FIG. 2 with multiway valves instead of standard valves;

FIG. 3a an embodiment with an additional balance chamber;

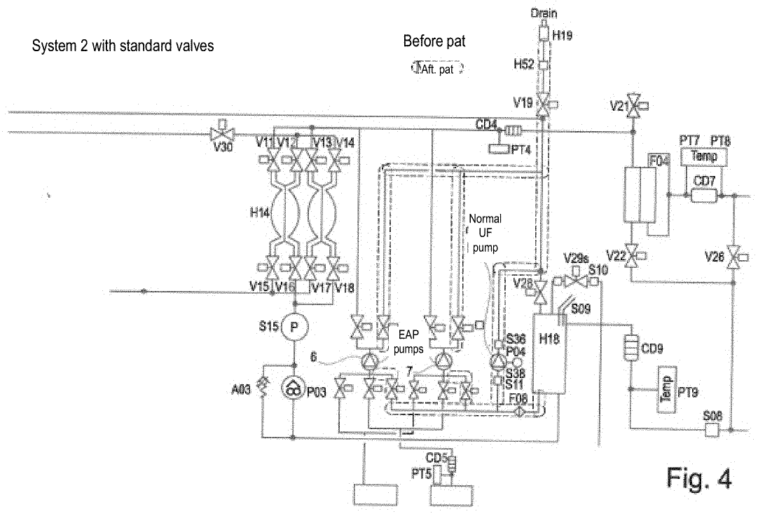

FIG. 4a flowchart that illustrates the use of pumps in accordance with the invention as part of a different embodiment of a blood treatment machine;

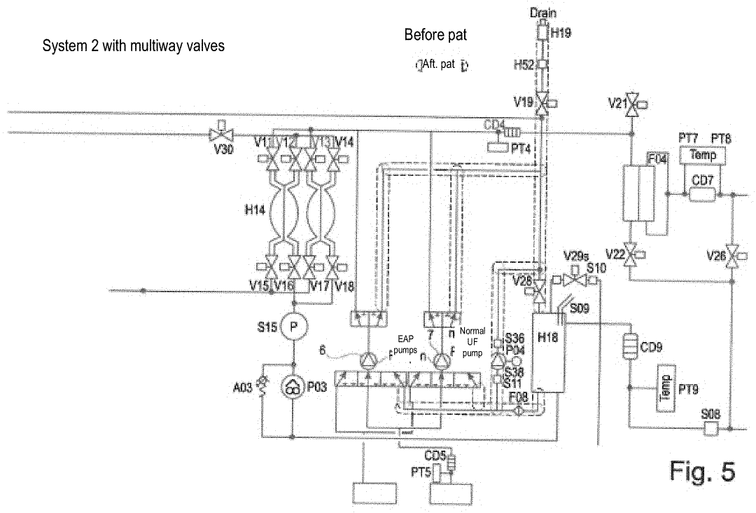

FIG. 5 shows the embodiment of FIG. 4 with multiway valves instead of standard valves;

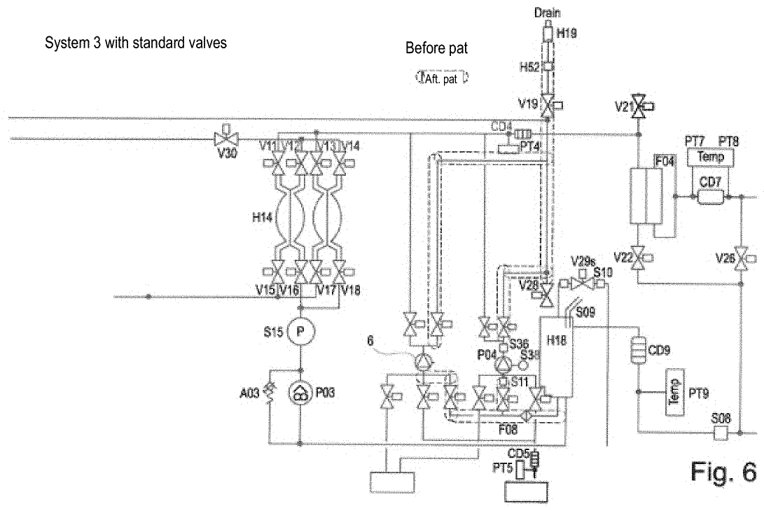

FIG. 6 a flowchart that illustrates the use of pumps in accordance with the invention as part of a different embodiment of a blood treatment machine;

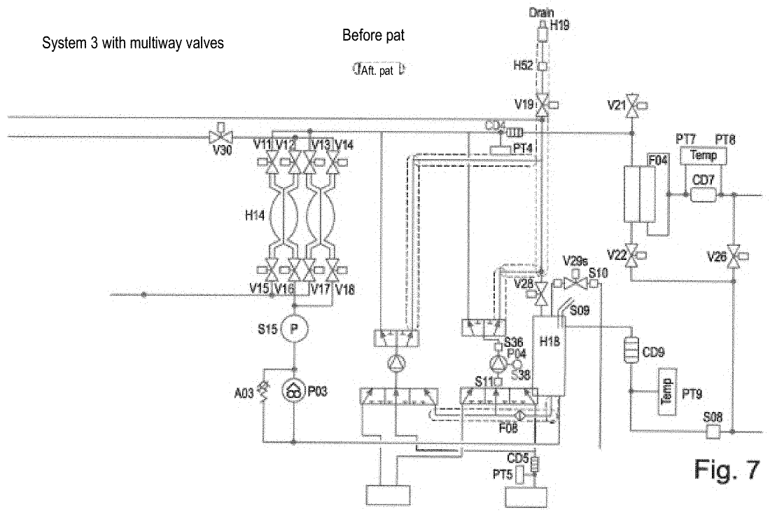

FIG. 7 snows the embodiment of FIG. 6 with multiway valves instead of standard valves;

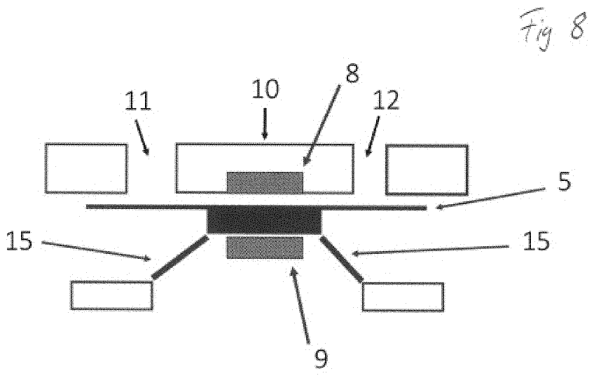

FIG. 8 shows an embodiment of a further pump in accordance with the invention; and

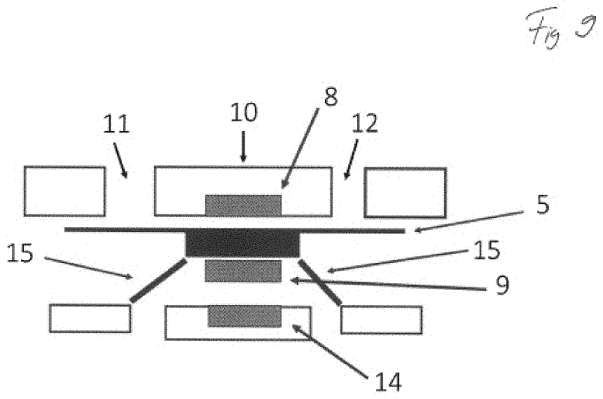

FIG. 9 shows an embodiment of a different pump in accordance with the invention.

The pump 1 shown in FIG. 1 has three stacks 2 of electroactive polymers arranged next to one another. The pressure generated by the stacks 2 of electroactive polymers is transmitted via a common connection plate 3 to a force bundling structure 4 whose area is smaller than the area of the connection plate 3. The pump shown in FIG. 1 can be used as a valve 1.

The force bundling structure 4 is connected to a pump membrane 5 that is moved up and down in accordance with the deformation of the stacks 2 of electroactive polymers. The longitudinal direction of the stacks 2 of electroactive polymers and the direction of the deformation of the stacks 2 are thus aligned in parallel with the direction of the pump strokes of the pump 1. In an optional variant, the force bundling structure 4 can also be designed as a stack of electroactive polymers. In this variant, however, it does not serve as an actuator that generates the stroke movement of the pump. This separate stack of electroactive polymers rather serves the more accurate self sensing of the stroke movement. For the force bundling, the electroactive polymer particularly advantageously here has a greater hardness or smaller elasticity than the electroactive polymers used for the stroke movement. An optional processing unit can more accurately determine the stroke that actually took place and the force that acts on the membrane in the stroke direction using the electrical measurements of the capacitances or potentials of all the electroactive polymers of the device.

The pump shown in FIG. 1 a additionally has a return element having two magnets 8, 9. The magnet 8 is installed at an upper element of the pump housing 10 or at the top of the pump chamber and the magnet 9 is connected to the pump membrane 5. It is ensured by the attraction force of the magnets 8, 9 that the membrane 5 is moved completely up to the upper abutment (top of the pump chamber) on every pump stroke. The conveying medium of the pump 1 flows into the pump chamber via an inlet 11 and exits the pump chamber via an outlet 12. In an alternative variant, the return element is alternatively or additionally designed as an electric capacitor having two capacitor plates 8, 9. It is achieved or facilitated by the electrostatic attraction of the plates to one another when the capacitor has a charge that the membrane 5 reaches the upper abutment of the pump. In this variant, monitoring can particularly advantageously be carried out by a monitoring of the electrical properties of the return element designed as a capacitor. If the membrane 5 is at the upper end abutment of the pump, a specific, known capacitance of the return element designed as a capacitor is adopted. The reaching of the end abutment can therefore be determined by the measurement of the capacitance or of other electrical properties (saturation current, impedance, saturation potential). A particularly precise pump can thereby be provided that yet further improves the high precision and measurement of the pump strokes by means of self sensing.

As shown in FIG. 1b, the magnets 8 and 13 interacting with the pump membrane 5 can also be installed at different locations on the pump housing 10. In the embodiment shown in FIG. 1b, the magnets 8 and 13 are installed at both sides in the side walls of the pump housing 10 close to a base of the pump chamber. In an alternative variant analog to the alternative described in connection with FIG. 1a, capacitor plates are provided instead of magnets or in addition to magnets so that a return element alternatively or additionally comprises one or more electric capacitors.

In FIG. 2 a flowchart is shown that illustrates the use of pumps in accordance with the invention as part of a blood treatment machine;

Dialysis is a process for blood purification that is used in patients who are affected by renal failure. Their kidneys are no longer able to filter toxins produced by the body from the blood. Further important processes in the regulation of the water and electrolyte metabolism of the patients are furthermore impaired.

To remove the toxins, the patient's blood is brought into contact with dialysis liquid via semipermeable hollow fiber membranes in a dialyzer. Substances, specifically toxins, electrolytes, and proteins, can diffuse to and fro between the liquids through this membrane. Since diffusion is a process driven by concentration and should prevent foreign bodies from diffusing into the patient's blood, the dialysis liquid, the so-called dialyzate, consists of ultrapure water.

Additional concentrates such as electrolyte concentrates and bicarbonate are additionally metered into the dialyzate to balance the electrolyte metabolism of the patient. This is conventionally done via the pumps P05 and P06 that are replaced by the present invention so that the pumps P05 and P06 are struck out in the Figures.

In a conventional blood treatment machine, the dialysis water is conveyed in the direction of the balancing chamber H14 by a pump via a heating chamber and an air separator. In addition to the dialysis water, acids and bases are added to the water and together form a physiological solution via the pumps P05 (concentrate pump) and P06 (bicarbonate pump).

It is technically ensured in the balancing chamber that the same amount of physiological solution is conveyed to the patient as liquid comes from the patient. An additional dialysis liquid filter F04 having a dead volume of e.g. approximately 300 ml is arranged between the balancing chamber and the dialyzer. This dead volume is relevant to the observation of flow peaks and their smoothing; this dead volume further acts as a kind of fluid store.

The physiological solution reaches the dialyzer, where it is used to purify the blood, from the dialysis liquid filter F04. After the purification of the blood, the dialysis liquid is again introduced into the balancing chamber H14 via a pump and finally enters into the drain.

In addition, an ultrafiltration pump P04 (UF pump) is installed in most blood treatment machines. It should remove additional water from the patient. The ultrafiltration pump is decoupled from the balancing by means of the balancing chamber H14 since it performs a targeted water removal that should not be corrected.

The concentrate and the bicarbonate are conventionally injected or metered into the line directly before the balancing chamber. A very high water pressure of approximately 1.8 bar is present at this position due to technical circumstances. This means that the pumps have to convey against this pressure, which represents a technical challenge.

An approach of a technical solution can thus comprise metering in downstream of the balancing chamber. A considerably smaller pressure is present after the balancing chamber.

A flowchart of an embodiment of the present invention is shown in FIGS. 2 and 3 in which both the concentrate and the bicarbonate are fed in by means of pumps in accordance with the invention (pumps having electroactive polymers, EAP pumps) after the balancing chamber H14 and directly before the dialysis liquid filter F04 that serve as a reservoir/mixing chamber.

A considerably smaller water pressure is present in this region than directly upstream of the balancing chamber H14. The volume is injected after or downstream of the balancing chamber (EAP pump 6 in the stored progression before/upstream of the patient “Before pat”) and is thus not considered in the balancing. The patient would thus be continuously subjected to excess water by the injected volume without any further measures. There is accordingly the necessity of removing this volume again.

This can be done either via the existing UF pump (P04) or via a further EAP pump (EAP pump 7 in the stored progression after/downstream of the patient “After pat”). Since the volume metered in by means of the EAP pump 6 is known, the same volume can easily be removed again.

The EAP pumps named here can be considered as volumetric metering systems. This means that a metering takes place in that a known pump space, that is a volume, is removed. Since the metered media are preferably liquids, that is are practically not compressible, the conveyed quantity—the dose—is clearly set by the volume or corresponds to the volume conveyed by a pump stroke or a specific number of pump strokes. In comparison with other methods of metering, the EAP pumps therefore bring about a higher reproduction accuracy as an intrinsic property, that is a higher accuracy on repeated procedures. To further improve this for the use of EAP pumps as metering and UF pumps here, a further balancing device 9 having a balancing chamber 9a that may be smaller van be interposed between the two pumps 5 and 6 and the line 8 into which metering takes place, as is shown schematically in FIG. 3a. In this embodiment, the dialyzer is marked by D, the patient by P, and the balancing device H14 comprises two balancing chambers 14a and 14b. In this further development, a check can be made redundantly by means of the balancing device 9 or the balancing chamber 9a that the metering by the EAP pumps was exact and increased safety can thereby be achieved. It is thus also ensured that exactly that metering volume is removed again that was previously added on the metering into the dialyzate circuit.

In embodiments in which a volumetric balancing chamber is arranged for the balancing of the metering between the metering pumps and the line into which the metering takes place, any other pumps can be used for the metering instead of EAP pumps such as peristaltic pumps, membrane pumps, gear pumps, centrifugal pumps—for example impeller pumps.

The properties (small strokes, high frequencies) of the pumps in accordance with the invention furthermore make it possible to combine different pump functions (e.g. the pumping of different solutions).

A blood treatment machine is thus generally conceivable in which a bicarbonate pump, a concentrate pump, and an ultrafiltration pump (UF pump) are designed as pumps in accordance with the invention. In this respect, the UF pump must be in a position to be able to remove both typical volumes to be removed and the metered electrolyte and bicarbonate volumes per treatment.

Alternatively, a single pump in accordance with the invention can also pump bicarbonate and concentrate and additionally a UF pump can be provided that is either a pump in accordance with the invention having electroactive polymers or is a conventional serial pump.

The functions of the pumping of bicarbonate and concentrate can also be combined in one pump in accordance with the invention; a UF pump and a balancing pump can additionally be provided that are each either a pump in accordance with the invention having electroactive polymers or a conventional serial pump. The object of the balancing pump in this respect comprises the removal of the volume added via the bicarbonate/concentrate pump. The UF pump therefore only has to convey the typical UF volume.

Alternatively, the functions of the bicarbonate pump, concentrate pump, and UF pump could also be combined in a single pump in accordance with the invention that is preferably present in the blood treatment device at least twice.

An embodiment will be explained in more detail by way of example in the following with respect to FIG. 4 or 5.

The EAP pumps 6 and 7 meter in concentrate and bicarbonate downstream of/after the balancing chamber H14, which enables an injection against a smaller pressure, and remove fluid for the balancing.

The first EAP pump 6 here takes over the metering/feeding of bicarbonate and concentrate, wherein, as shown in FIG. 5, it is possible to alternate between the two fluids by a multiway valve. The switchover of the fluids can also take place by regular valves (see FIG. 4). Since the metering/feeding takes place downstream of the balancing chamber, but upstream of the patient (see flow path “Before pat”), the fluid amount fed in has to be removed again to prevent an excess water supply to the patient. Fluid is therefore removed from the system after the patient (see flow path “After pat”), which is taken over by the second EAP pump 7. The original UF pump P04 remains in the system here.

In general, both pumps 6 and 7 can also take over both the feed and the removal of liquid.

A pump always has certain tolerances for technical production reasons and thus has a certain inaccuracy in the pumping of a defined volume. This has the result that, on the one hand, errors occur in the metering (metering errors) and in the removal of fluid. Considered overall, balancing defects thereby occur.

To balance these volume errors (metering and balancing errors) of the individual pumps, it is furthermore possible to interconnect the pumps via a valve circuit such that the functions of the pumps can be swapped by a switchover. This enables a compensation of the error. A control unit must be provided for this purpose that is adapted to correspondingly control the pumps.

In general, the EAP pump used or a plurality of EAP pumps used can be installed with the associated valves in a pump unit. In this respect, the metering pump(s) and the valves are preferably installed on one unit and can be operated by a central control. It can also include valves that are required for the feeding of fluids downstream of the balancing chamber.

It is possible by the use of a separate central control unit for the valves and the pump to operate the circuit of the pump unit completely separately from the control of a blood treatment machine. This separate control unit, for example, has its own CPU, which makes possible considerably shorter processing times in comparison with the use of the machine software. It is possible to reach higher pump frequencies by this reduced processing time.

It is thereby possible to utilize the specific characteristics of the EAPs (comparatively small stroke, but high frequency). In this respect, the desired conveying rate is necessary as the only input parameter on the machine software side; the required frequency and the required pump volume are then preferably calculated by the separate control unit.

A further embodiment of a blood treatment machine is shown in FIG. 6 that has a pump 6 in accordance with the invention and an ultrafiltration pump P04. The pump 6 in accordance with the invention pumps both concentrate and bicarbonate and the ultrafiltration pup P04 is preferably likewise designed as an EAP pump in accordance with the invention, but can also be a different kind of pump.

FIG. 8 shows a further pump in accordance with the invention. The pump has a housing 10 in whose upper abutment a polymagnet or a programmed magnet 8 has been placed. A further polymagnet or programmed magnet 9 is provided via the pump membrane 5 spaced apart from the polymagnet or programmed magnet, said further magnet 9 being connected to the housing of the pump via a COP electroactive polymer 15.

If the COP 15 is deformed, the polymagnet 9 is moved toward the polymagnet 8. If a specific distance between the polymagnets 8 and 9 has been reached, they repel one another and the polymagnets 8 and 9 move away from one another. The pump membrane 5 carries out this movement subsequently so that the pump membrane 5 is moved to convey medium by the deformation of the COP 15 and the movement of the polymagnets 8 and 9 toward and away from one another.

As shown in FIG. 9, a further polymagnet 14 can additionally be installed. The polymagnet 9 arranged between the polymagnets 8 and 14 is thus moved to and fro between the polymagnets 8 and 14, with the attraction or repulsion of the polymagnets having the effect that the polymagnet 9 reliably moves to and fro between an upper abutment and a lower abutment.

1. A pump (1) for a medical device, in particular a blood treatment device, preferably a dialysis machine, comprising
a pump actuator having at least one electroactive polymer, wherein the pump actuator has a trigger element, a displacement element, and a return element, with the return element being configured such that the displacement element can be moved to a defined position after an actuation of the trigger element.

2. A pump (1) in accordance with claim 1, characterized in that the return element has at least one spring, one magnet (8, 9, 13, 14), or one capacitor and the return element brings the trigger element into a defined position that is a pump position or the return element brings the trigger element into a defined position that is opposite to the pump position.

3. A pump (1) in accordance with claim 1, characterized in that the pump actuator, preferably the displacement element and/or the trigger element, has a plurality of layers, preferably a stack (2), of electroactive polymers, in particular dielectric electroactive polymers.

4. A pump (1) in accordance with claim 1, wherein the pump actuator has electrodes arranged in ring form, respectively having a silicone layer that is disposed between the electrodes and that are preferably configured as the trigger element.

5. A pump (1) in accordance with claim 1, characterized in that the pump actuator has a membrane (5) and the displacement element has a plurality of layers, preferably a stack (2), of electroactive polymers, and the membrane (5) can be moved from a starting position into a pump position by means of the electroactive polymers.

6. A pump (1) in accordance with claim 1, wherein the trigger element has a plurality of layers, preferably a stack (2), of electroactive polymers or electrodes arranged in ring form, respectively having a silicone layer disposed between the electrodes.

7. A pump (1) in accordance with claim 1, wherein the displacement element and/or the return element and/or the trigger element are formed as one element in part.

8. A pump (1) in accordance with claim 1, characterized in that the return element has one or more magnets and the at least one magnet (8, 9, 13, 14) has at least 8 individual poles, preferably more than 10 individual poles, even more preferably more than 14 individual poles.

9. A pump (1) in accordance with claim 1, characterized in that the return element comprises at least one capacitor, with the capacitor being charged by the released energy on a dilatation of the electroactive polymer and with the released energy being able to be used for a contraction of the electroactive polymer on a discharge of the capacitor.

10. A pump cabin (1) in accordance with claim 1, wherein the return element is at least one capacitor, the trigger element is an electroactive polymer, and the displacement element is at least one spring, at least one magnet, or at least one capacitor, each being connected to a movable membrane (5), preferably further comprising an electric energy storage device that can store the energy of the capacitor and/or of the dielectric elastomer.

11. A metering unit, in particular a metering unit for a blood treatment device, having a pump (1) in accordance with claim 1.

12. A medical device, in particular a blood treatment machine, having a pump (1) and/or a metering unit in accordance with claim 1.

13. Use of a pump (1) in accordance with claim 1 in a medical device, in particular a blood treatment apparatus, preferably a dialysis machine.

14. A method of pumping fluid for a medical device, in particular a blood treatment apparatus, in particular a dialysis machine, having a pump (1) in accordance with claim 1, wherein the pump (1) has a pump space for conveying fluid, comprising
triggering the trigger element by changing an electric voltage;
moving the displacement element from a starting position into a pump position so that fluid is displaced from a pump space; and
returning the displacement element from the pump position into the starting position or returning the displacement element into the pump position after leaving the starting position.

15. A method in accordance with claim 1, wherein the return element has a capacitor; and wherein the triggering of the trigger element takes place by a discharge of the capacitor and a return of the displacement element takes place by a charging of the capacitor; and/or wherein at least a portion of the electrical charge or energy of the capacitor flows between the return element and the displacement element.
